(19) United States
(12) Patent Application Publication
(10) Pub. No.: US 2015/0178957 A1
(43) Pub. Date: Jun. 25, 2015

(54) ITERATIVE RECONSTRUCTION FOR SPECTRAL CT BASED UPON COMBINED DATA OF FULL VIEWS OF INTENSITY DATA AND SPARSE VIEWS OF SPECTRAL DATA

(71) Applicants: TOSHIBA MEDICAL SYSTEMS CORPORATION, Otawara-shi (JP); KABUSHIKI KAISHA TOSHIBA, Tokyo (JP)

(72) Inventor: Yu ZOU, NAPERVILLE, IL (US)

(21) Appl. No.: 14/137,254

(22) Filed: Dec. 20, 2013

(51) Int. Cl. G06T 11/00; A61B 6/03; A61B 6/00

(52) U.S. Cl. CPC G06T 11/005; A61B 6/482; A61B 6/032; A61B 6/4241; G06T 2207/10081

(57) ABSTRACT

Full views of the intensity data and the sparse views of the spectral data are obtained so as to combine the two sets of the acquired data. An image is reconstructed using an iterative reconstruction algorithm to minimize a predetermined cost function based upon the combined two sets of full views of the intensity data and the sparse views of the spectral data.

### FIELD OF THE INVENTION

The current invention is generally related to spectral computer tomography (CT) image processing, and more particularly related to iterative reconstruction of CT images based upon the combined data of full views of intensity data and sparse views of spectral data.

### BACKGROUND OF THE INVENTION

The x-ray beam in most computer tomography (CT) scanners is generally polychromatic. Yet, most of the currently used CT scanners generate images based upon data according to the energy integration nature of the detectors. These conventional detectors are called energy integrating detectors for acquiring energy integration X-ray data that cannot provide spectral information. On the other hand, photon counting detectors are configured to acquire the spectral nature of the x-ray source rather than the energy integration nature in the acquired data. To obtain the spectral nature of the transmitted X-ray, the photo counting detector splits the x-ray beam into its component energies or spectrum bins and counts a number of photons in each of the bins. The use of the spectral nature of the x-ray source in CT is often referred to as spectral CT. Since spectral CT involves the detection of transmitted X-ray at two or more energy levels, spectral CT generally includes dual-energy CT by definition.

Spectral CT is advantageous over conventional CT in certain aspects. Spectral CT offers the additional clinical information inherent in the full spectrum of an x-ray beam. For example, spectral CT enhances in discriminating tissues, differentiating between materials such as tissues containing calcium and iodine or enhancing the detection of smaller vessels. Among other advantages, spectral CT is also expected to reduce beam hardening artifacts. Spectral CT is expected to increase accuracy in CT numbers independent of scanners.

Prior art attempts for spectral CT unfortunately involve tradeoffs while trying to solve issues such as beam hardening, temporal resolution, noise balance, and inadequate energy separation. For example, dual source solutions are good for noise balance and energy separation but are not so good in some clinical applications for correcting beam hardening and improving temporal resolution. Fast kV-switching has the potential for good beam hardening correction and good temporal resolution although the noise balance might require a tradeoff with temporal resolution and inadequate energy separation might affect the precision of the reconstructed spectral images. Nonetheless, when used in the right clinical situations, prior art solutions can successfully improve diagnosis. On the other hand, spectral imaging with photon counting detectors has the potential for solving all four issues without tradeoffs as well as more advanced spectral techniques such as precise material characterization through k-edge imaging.

Prior art has also attempted to replace the conventional integrating detectors by the photon counting detectors in implementing spectral CT. In general, photon counting detectors are costly and have performance constraints under high flux x-rays. Although at least one experimental spectral CT system has been reported, the costs of high-rate photon counting detectors are prohibitive for a full-scale implementation. Despite some advancement in the photon counting detector technology, the currently available photon counting detectors still require solutions to implementation issues such as polarization due to space charge build-up, pile-up effects, scatter effects, spatial resolution, temporal resolution and dose efficiency.

Spectral CT is currently limited to dual energy approaches such as dual source CT, dual layer detector CT and fast-kV switching CT. In this regard, true spectral information beyond dual energy is not advantageously utilized in general purpose clinical CT. On the other hand, a true spectral CT system appears to face the above described issues related to the energy differentiating photon counting detectors.

For the above reasons, it is still desired to invent CT systems and methods of improving the use of spectral data as acquired by the photon counting detectors possibly in combination with energy integrated data as acquired by the energy integration detectors.

### BRIEF DESCRIPTION OF THE DRAWINGS

FIG. 1 is a diagram illustrating one X-ray CT apparatus or scanner according to the current invention including a gantry and other devices or units.

FIG. 2A is a diagram illustrating one embodiment of the intensity data acquiring device of the CT scanner system according to the current invention.

FIG. 2B is a diagram one embodiment including the intensity data acquiring device and the spectral data acquiring device of the CT scanner system according to the current invention.

FIG. 3 is a flow chart illustrating steps or acts involved in a process of reconstructing an image based upon full views of the intensity data and sparse views of the spectral data according to the current invention.

FIG. 4A is a basis image for bone in a torso phantom based upon the full integration data of 140 kVP and 1200 views and the sparse spectral data of 100 kVP and 75 views.

FIG. 4B is a basis image for water in the torso phantom based upon the full integration data of 140 kVP and 1200 views and the sparse spectral data of 100 kVP and 75 views.

FIG. 4C is a monochromatic image at 50 keV of the torso phantom of FIGS. 4A and 4B.

FIG. 4D is a monochromatic image at 75 keV of the torso phantom of FIGS. 4A and 4B.

FIG. 5A is a basis image for bone in a torso phantom based upon the full integration data of 140 kVP and 1200 views and the sparse spectral data of 100 kVP and 150 views.

FIG. 5B is a basis image for water in the torso phantom based upon the full integration data of 140 kVP and 1200 views and the sparse spectral data of 100 kVP and 150 views.

FIG. 5C is a monochromatic image at 50 keV of the torso phantom of FIGS. 5A and 5B.

FIG. 5D is a monochromatic image at 75 keV of the torso phantom of FIGS. 5A and 5B.

FIG. 6A is a basis image for bone in a head phantom based upon the full integration data of 140 kVP and 1200 views and the sparse spectral data of 100 kVP and 75 views.

FIG. 6B is a basis image for water in the head phantom based upon the full integration data of 140 kVP and 1200 views and the sparse spectral data of 100 kVP and 75 views.

FIG. 6C is a monochromatic image at 50 keV of the head phantom of FIGS. 6A and 6B.

FIG. 6D is a monochromatic image at 75 keV of the head phantom of FIGS. 6A and 6B.

FIG. 7A is a basis image for bone in a head phantom based upon the full integration data of 140 kVP and 1200 views and the sparse spectral data of 100 kVP and 150 views.

FIG. 7B is a basis image for water in the head phantom based upon the full integration data of 140 kVP and 1200 views and the sparse spectral data of 100 kVP and 150 views.

FIG. 7C is a monochromatic image at 50 keV of the head phantom of FIGS. 7A and 7B.

FIG. 7D is a monochromatic image at 75 keV of the head phantom of FIGS. 7A and 7B.

### DETAILED DESCRIPTION OF THE PREFERRED EMBODIMENT(S)

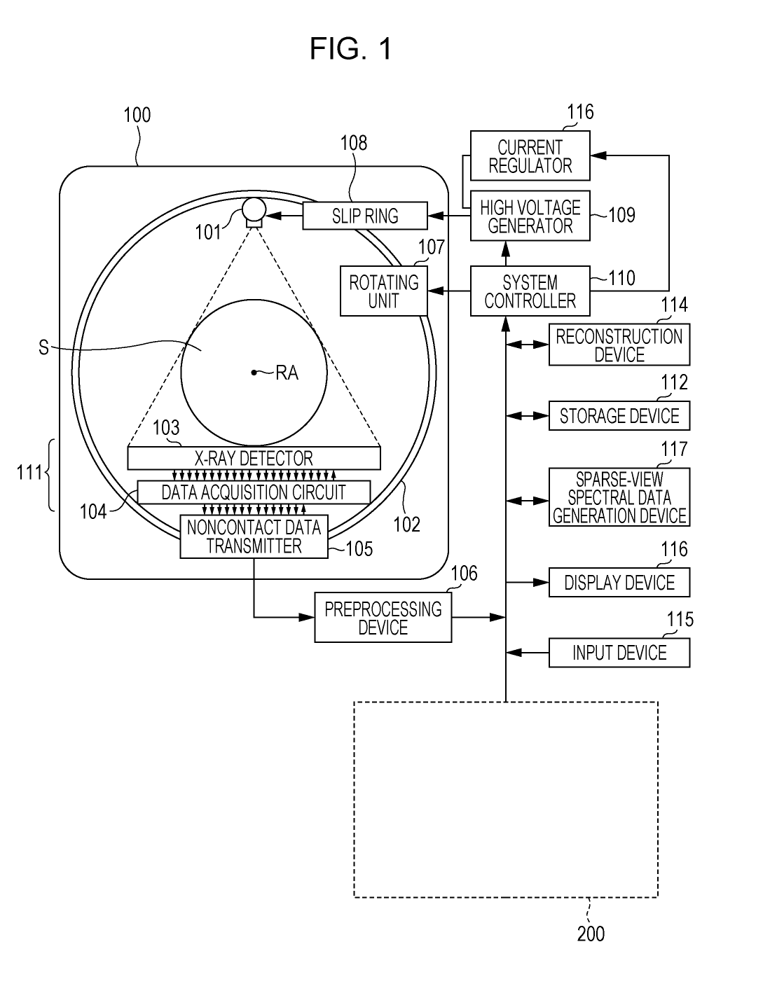

Referring now to the drawings, wherein like reference numerals designate corresponding structures throughout the views, and referring in particular to FIG. 1, a diagram illustrates one X-ray CT apparatus or scanner according to the current invention including a gantry 100 and other devices or units. The gantry 100 is illustrated from a side view and further includes an X-ray tube 101, an annular frame 102 and a multi-row or two-dimensional array type X-ray detector 103. The X-ray tube 101 and X-ray detector 103 are diametrically mounted across a subject S on the annular frame 102, which is rotatably supported around a rotation axis RA. A rotating unit 107 rotates the frame 102 at a high speed such as 0.4 sec/rotation while the subject S is being moved along the axis RA into or out of the illustrated page.

The multi-slice X-ray CT apparatus further includes a high voltage generator 109 that generates a tube voltage to be applied to the X-ray tube 101 through a slip ring 108 so that the X-ray tube 101 generates X ray. The X rays are emitted towards the subject S, whose cross sectional area is represented by a circle. The X-ray detector 103 is located at an opposite side from the X-ray tube 101 across the subject S for detecting the emitted X rays that have transmitted through the subject S. The X-ray detector 103 further includes individual detector elements or units that are conventional integrating detectors.

Still referring to FIG. 1, the X-ray CT apparatus or scanner further includes other devices for processing the detected signals from X-ray detector 103. A data acquisition circuit or a Data Acquisition System (DAS) 104 converts a signal output from the X-ray detector 103 for each channel into a voltage signal, amplifies it, and further converts it into a digital signal. The X-ray detector 103 and the DAS 104 are configured to handle a predetermined total number of projections per rotation (TPPR) that can be at the most 900 TPPR, between 900 TPPR and 1800 TPPR and between 900 TPPR and 3600 TPPR.

The above described data is sent to a preprocessing device 106, which is housed in a console outside the gantry 100 through a non-contact data transmitter 105. The preprocessing device 106 performs certain corrections such as sensitivity correction on the raw data. A storage device 112 then stores the resultant data that is also called projection data at a stage immediately before reconstruction processing. The storage device 112 is connected to a system controller 110 through a data/control bus, together with a reconstruction device 114, an input device 115, a display device 116 and a scan plan support apparatus 200. The scan plan support apparatus 200 includes a function for supporting an imaging technician to develop a scan plan.

The detectors are either rotated or fixed with respect to the patient among various generations of the CT scanner systems. The above described CT system has one exemplary third-generation geometry in which the X-ray tube 101 and the energy integrating X-ray detector 103 are diametrically mounted on the annular frame 102 and are moved around the subject S as the annular frame 102 is rotated about the rotation axis RA.

In one embodiment according to the current invention, as illustrated in FIG. 1, the energy integrating detector 103 and the radiation emitting source or X-ray tube 101 are used to acquire full views of intensity data of an object or a subject S and then to acquire sparse views of spectral data of the object using the full views of intensity data before reconstructing an image of the object based upon the full views of the intensity data and the sparse views of the spectral data.

In another embodiment according to the current invention, although it is not illustrated in the diagram of FIG. 1, a fourth-generation geometry has a spectral data acquiring device such as energy differentiating detectors that are fixedly placed around the patient S. For example, a predetermined number of energy differentiating detectors such as photon counting detectors and semiconductor direct conversion detectors are fixedly placed along the subject S along a predetermined path. In one implementation, the photon counting detectors are sparsely mounted inside or along the predetermined path, which is located between a first trajectory of the energy integrating detector 103 and a second trajectory of the radiation emitting source or X-ray tube 101.

FIG. 1 illustrates the use of the rotating energy integrating detector 103 in acquiring projection data in a third-generation geometry for reconstructing an image of the object based upon the full views of the intensity data and the sparse views of the spectral data. One embodiment initially obtains two data sets of the full views of the intensity data respectively with a high energy level such as 135 kV and a low energy level such as 80 kV on a third-generation scanner. The embodiment further includes a sparse-view spectral data generation device 117 for generating or simulating sparse views of the spectral data based upon the full views of the intensity data that has been acquired by an intensity data acquiring device such as the rotating energy integrating detector 103 in a third-generation geometry. The sparse-view spectral data generation device 117 selects one view for every predetermined number of N views such as N=8 in the low energy data and discard the rest. Thus, the sparse-view spectral data generation device 117 generates the sparse pairs of low/high energy data that mimics the spectral data from the sparse photon counting detectors on a fourth-generation geometry. The reconstruction device 114 utilizes the full high energy data from a third-generation scanner and the generated sparse spectral data in reconstructing an image.

As will be further described later, the above described embodiments are mere examples and are not limited in many aspects. For example, although the above described embodiment includes the sparse-view spectral data generation device 117, the sparse-view spectral data generation device 117 is not necessary to practice the claimed invention. In other embodiments, the full views of the intensity data and the sparse views of the spectral data are optionally obtained simultaneously, sequentially or independently, using a single scanner or a plurality of scanners. Although the energy differentiating detectors and the energy integrating detector are not necessarily on the same scanner, the full views of the intensity data and the sparse views of the spectral data are simultaneously acquired from a single scanner for their spatial and temporal resolution in one embodiment.

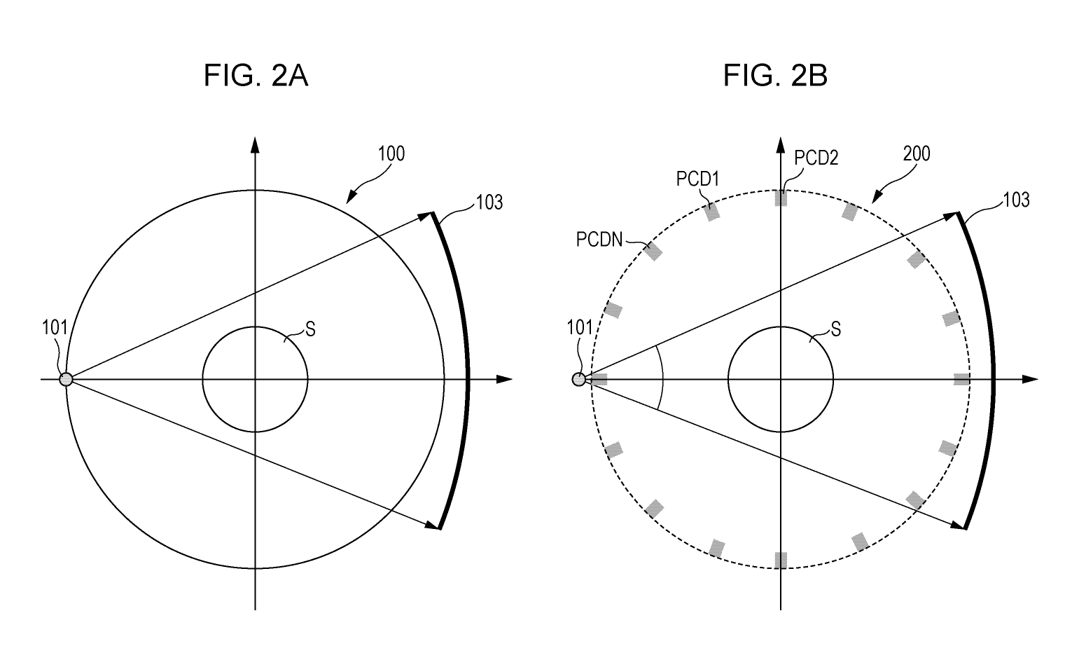

Now referring to FIG. 2A, a diagram illustrates one embodiment of the intensity data acquiring device of the CT scanner system according to the current invention. The CT system has one exemplary third-generation geometry in which the X-ray tube 101 and the energy integrating X-ray detector 103 are diametrically mounted on a predetermined annular frame and are moved around the subject S as the annular frame is rotated about a predetermined rotation axis. During the rotation, the X-ray tube 101 travels a predetermined trajectory 100 while keeping a diametrically opposed position with respect to the energy integrating X-ray detector 103. The detector 103 detects the transmitted X-ray and generates an energy integration signal in full views while the X-ray tube 101 emits X-ray at a predetermined energy level. The intensity data acquiring device acquires full views of intensity data of the subject S or an object as the X-ray tube 101 and the energy integrating X-ray detector 103 travel around the subject. Two sets of full views of intensity data may be optionally acquired at two different energy levels so that the intensity data is used for generating simulated sparse views of spectral data.

In another embodiment according to the current invention, a predetermined number of energy differentiating detectors such as photon counting detectors and semiconductor direct conversion detectors are fixedly placed along an object along a predetermined path 200. As illustrated in FIG. 2B, the photon counting detectors PCD1 through PCDN are sparsely mounted inside or along the predetermined path 200, which is located inside a first trajectory of the energy integrating detector 103 and a second trajectory of the radiation emitting source or X-ray tube 101. In the illustrated embodiment, the trajectory of the source tube 101 is illustrated to have a larger diameter so that a predetermined beam angle encompasses a predetermined portion of the predetermined path 200. In another embodiment, the trajectory of the source tube 101 may have a certain diameter that a predetermined beam angle may encompass a smaller portion of the predetermined path 200.

In the above described relative spatial relationship, the radiation emitting source 101 is moved along the predetermined path outside the first path of the fixedly placed photon counting detectors while continuously emitting radiation towards the object. In this regard, the X-ray that is emitted from the source 101 towards the subject S, and some radiation reaches the energy integrating detector 103 after transmitted through the subject S while other radiation also reaches a certain portion of the energy differentiating detectors, whose detection surface is located at a certain angle with respect to the source 101. Spectral data is detected at the energy differentiating detectors, which are sparsely fixed with respect to the source 101. Energy integration data is detected at the energy integrating detector 103, which is rotated with the source 101. Thus, both the energy integrating detector 103 and the energy differentiating detectors PCDs continuously acquire a combination of the data for later reconstructing an image at the reconstruction device 114. In any case, FIG. 2B illustrates a combined use of the rotating energy integrating detector 103 and the fixedly mounted energy differentiating detectors for respectively acquiring full views of the intensity data and sparse views of the spectral data.

An alternative embodiment includes the rotating energy integrating detector 103 and the fixedly mounted energy differentiating detectors in separate scanners or separate housings of a single scanner. That is, the full views of the intensity data and the sparse views of the spectral data are acquired sequentially or independently, but not simultaneously. Although the alternative embodiment may be optionally implemented to have the sequential or independent scans, the acquired data may have temporal and or spatial correction before reconstructing an image.

As will be further illustrated, the above described embodiment is a mere example and is not limited in many aspects. For example, although a certain spatial relationship of the trajectories or paths are disclosed among the source 101, the energy differentiating detectors PCDs and the energy integrating detector 103, the spatial relationship is relative and not limited to a particular relation as illustrated in the diagram. Lastly, although a single pair of the energy integrating detector 103 and the radiation source 101 is illustrated in the embodiment, an additional pair of the energy integrating detector 103 and the radiation source 101 is incorporated in another embodiment according to the current invention.

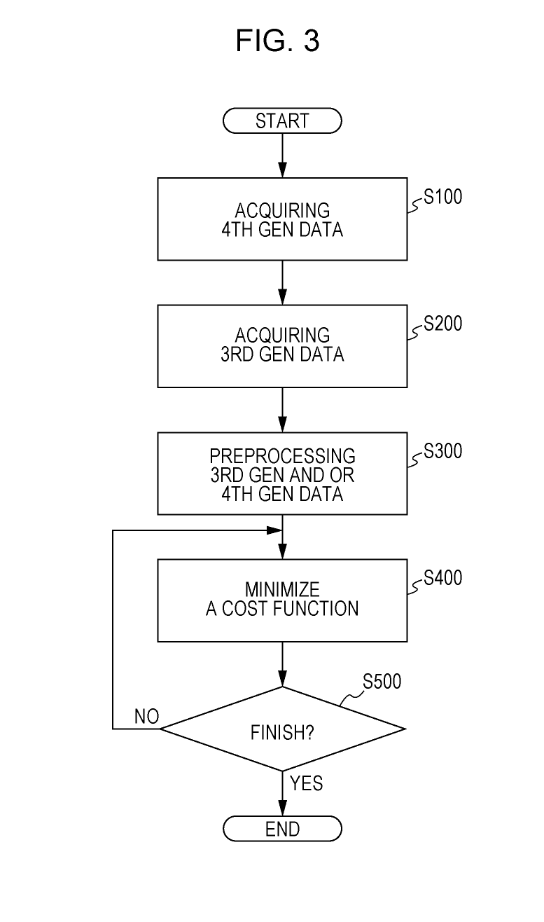

Now referring to FIG. 3, a flow chart illustrates steps or acts involved in a process of reconstructing an image based upon full views of the intensity data and sparse views of the spectral data according to the current invention. Ultimately, the full views of the intensity data and the sparse views of the spectral data are both used in generating an improved image according to the current invention. In this regard, the process of the reconstructing an image does not concern itself as to how the full views of the intensity data and the sparse views of the spectral data are acquired prior to the image construction. That is, the full views of the intensity data and the sparse views of the spectral data are simultaneously acquired using a single scanner in one process according to the current invention. In another process, the full views of the intensity data and the sparse views of the spectral data are sequentially or independently acquired using multiple scanners.

There are also other variations as to a number of views in the acquired data. In one method of reconstructing an image, the sparse views include as few as approximately 75 views for the spectral data according to the current invention. In one method of reconstructing an image, the full views include as many as approximately 1200 views for the intensity data according to the current invention. A number of views is not limited to the above exemplary numbers and has a certain range. That is, the above exemplary numbers are a mere illustration of practicing the current invention. The number of views optionally varies depending upon the clinical conditions and the scanner parameters.

Still referring to FIG. 3, the data are acquired or made available in the initial steps or acts according to the current invention. In a step S100, the sparse views of the spectral data or the fourth-generation data (4^{th} Gen Data) is acquired or obtained. That is, the spectral data is made available in a predetermined number of energy levels or bins based upon a predetermined number of sparse views. The fourth-generation data is optionally either being acquired during the step S100. Alternatively, the previously acquired fourth-generation data is made available in the step S100. By the same token, in a step S200, the full views of the intensity data or the third-generation data (3^{rd} Gen Data) is acquired or obtained. That is, the intensity data is made available based upon a predetermined number of full views. The third-generation data is optionally either being acquired during the step S200. Alternatively, the previously acquired third-generation data is made available in the step S200. The illustrated process does not necessarily require that the step S100 precedes the step S200. In this regard, the step S100 and the step S200 are optionally performed in a simultaneous or parallel manner. Furthermore, the step S100 and the step S200 are optionally performed in a reversed order from the illustrated process in the flow chart. On the other hand, it is essential that the full views of the intensity data and the sparse views of the spectral data are both available after the step S200 before a next step in a preferred process of reconstructing an image according to the current invention.

In a step S300, the intensity data and or the spectral data are optionally preprocessed to form a cost function according to the current invention. In general, the cost function includes a system matrix respectively for the 3^{rd} and 4^{th} Gen Scanners, basis images, the intensity data, spectral data, a regularization term and a beam hardening correction. In one embodiment of the process according the current invention, the regularization is optionally turned off or excluded. In one embodiment, the system matrix for the 3^{rd} and 4^{th} Gen Scanners is in polar coordinates.

One exemplary cost function is provided in Equations (1), (2) and (3) below:

$$\begin{matrix}ψ ( c ) = {\sum }_{jn} \frac{1}{{σ}_{jn}^{2}} {( {l}_{n} ( j ) - {l}_{n}^{( M )} ( j ) )}^{2} + {\sum }_{j} \frac{1}{{σ}_{j}^{2}} {( {\sum }_{n = 1}^{N} {L}_{n} ( j ) {\overline{μ}}_{nM} - {g}_{M} ( j ) - {g}_{M}^{( BH )} ( L ) )}^{2} + wV ( c ) & ( 1 ) \\ wherein \\ {l}_{n} ( j ) = {\sum }_{i} {a}_{ji} {c}_{n} ( i ) & ( 2 ) \\ {L}_{n} ( j ) = {\sum }_{i} {A}_{ji} {c}_{n} ( i ) & ( 3 )\end{matrix}$$

where c is a basis image vector, σⱼₙ² variance of lₙ^{(M)}(j) while σⱼ² is variance of g_{M}(j), μ̅_{nM} is an average linear attenuation coefficient over spectrum of 3rd gen for basis n, aⱼᵢ is a system matrix for a 4^{th}-Gen scanner for acquiring the sparse views of the spectral data, Aⱼᵢ is a system matrix for a 3^{rd}-Gen scanner for acquiring the full views of the intensity data, cₙ (i) is material basis images, V(c) is a regularization term, g_{M}(j) is the full views of the intensity data (measured 3^{rd} Gen data), g_{M}^{(BH)}(L) is a beam-hardening correction term with L being a material length vector of the basis material, lₙ^{(M)}(j) is material length for basis n along a ray j from the sparse views of the spectral data after decomposition (measured 4^{th} Gen data after decomposition). lₙ(j) is a re-projected material length for the 4^{th} Gen geometry while Lₙ(j) is a re-projected material length for the 3^{rd} Gen geometry. In one embodiment, a weight w for the regularization term is set to zero (w=0) for turning off the regularization term as used in one embodiment of the current invention.

In addition to the above disclosed cost function, there are other cost functions that are optionally used in a process of reconstructing an improved image based upon the measured 3rd Gen data and the measured 4th Gen data according to the current invention. Another exemplary cost function involves no data decomposition of the 3^{rd} Gen data or the 4^{th} Gen data and is provided in Equations (3), (4) and (5) below:

$$\begin{matrix}ψ ( c ) = {\sum }_{jm} \frac{1}{{σ}_{jm}^{2}} {( {g}_{m} ( j ) - {\sum }_{n = 1}^{N} {l}_{n} ( j ) {\overline{μ}}_{nm} + {g}_{M}^{( BH )} ( l ) )}^{2} + {\sum }_{j} \frac{1}{{σ}_{j}^{2}} {( {\sum }_{n = 1}^{N} {L}_{n} ( j ) {\overline{μ}}_{nm} - {g}_{M} ( j ) - {g}_{M}^{( BH )} ( L ) )}^{2} + w ( c ) V ( c ) & ( 3 ) \\ wherein \\ {l}_{n} ( j ) = {\sum }_{i} {a}_{ji} {c}_{n} ( i ) & ( 4 ) \\ {L}_{n} ( j ) = {\sum }_{i} {A}_{ji} {c}_{n} ( i ) & ( 5 )\end{matrix}$$

where c is a basis image vector, σⱼₙ² variance of g_{M}(j) while σⱼ² is variance of g_{M}(j). μ̅_{nM} an average linear attenuation coefficient over spectrum of 3rd gen for basis n, aⱼᵢ is a system matrix for a 4^{th}-Gen scanner for acquiring the sparse views of the spectral data, Aⱼᵢ is a system matrix for a 3^{rd}-Gen scanner for acquiring the full views of the intensity data, cₙ (i) is material basis images, V(c) is a regularization term, gₘ(j) is measured projection data or spectral data for energy bin m along ray j (measured 4^{th} Gen data), g_{M}(j) is the full views of the intensity data (measured 3^{rd} Gen data), g_{M}^{(BH)}(L) is a beam-hardening correction term with L being a material length vector of the basis material. lₙ(j) is a re-projected material length for the measured 4^{th} Gen data while Lₙ(j) is a re-projected material length for the measured 3^{rd} Gen data. In one embodiment, a weight w for the regularization term is set to zero (w(c)=0) for turning off the regularization term as used in one embodiment of the current invention.

Another exemplary cost function is provided in Equations (6), (7) and (8) below. The cost function (6) involves 4^{th} Gen data pre-decomposition and weight with a predetermined information matrix such as Fisher information matrix.

$$\begin{matrix}ψ ( c ) = {\sum }_{{jnn}^{′}} {I}_{{nn}^{′}} ( j ) ( {\sum }_{i} {a}_{ji} {c}_{n} ( i ) - {l}_{n}^{( M )} ( j ) ) ( {\sum }_{{i}^{′}} {a}_{ji} {c}_{{n}^{′}} ( {i}^{′} ) - {l}_{{n}^{′}}^{( M )} ( j ) ) + {\sum }_{j} \frac{1}{{σ}_{j}^{2}} {( {\sum }_{n = 1}^{N} {L}_{n} ( j ) {\overline{μ}}_{nM} - {g}_{M} ( j ) - {g}_{M}^{( BH )} ( L ) )}^{2} + wV ( c ) & ( 6 ) \\ wherein \\ {l}_{n} ( j ) = {\sum }_{i} {a}_{ji} {c}_{n} ( i ) & ( 7 ) \\ {L}_{n} ( j ) = {\sum }_{i} {A}_{ji} {c}_{n} ( i ) & ( 8 )\end{matrix}$$

Fisher information matrix is defined by the following terms:

$${I}_{{nm}^{′}} ( j ) = {\sum }_{E} \frac{1 \partial {N}_{j} ( E ) \partial {N}_{j} ( E )}{{N}_{j} ( E ) \partial {l}_{n} ( j ) \partial {l}_{{n}^{′}} ( j )}$$

Nⱼ(E) is a photon count in an energy bin E for a ray path j.
where c is a basis image vector, σⱼ² is variance of g_{M}(j), μ̅_{nM} is an average linear attenuation coefficient over spectrum of 3rd gen for basis n, a′ⱼᵢ is a system matrix for a 4^{th}-Gen scanner for acquiring the sparse views of the spectral data, Aⱼᵢ is a system matrix for a 3^{rd}-Gen scanner for acquiring the full views of the intensity data, c^{′}ₙ(i^{′}) is a material basis image for material n at a pixel i, V (c) is a regularization term, g_{M} (j) is the full views of the intensity data (measured 3^{rd} Gen data), g_{M}^{(BH)}(L) is a beam-hardening correction term with L being a material length vector of the basis material, lₙ^{(M)}(j) is material length for basis n along a ray j from the sparse views of the spectral data after decomposition (measured 4^{th} Gen data after decomposition). lₙ^{′(M)(j)} is a basis line integral in 4th gen geometry for material n and ray j from Measurement (after data domain decomposition). lₙ(j) is a re-projected material length for the measured 4^{th} Gen data while Lₙ(j) is a re-projected material length for the measured 3^{rd} Gen data. In one embodiment, a weight w for the regularization term is set to zero (w=0) for turning off the regularization term as used in one embodiment of the current invention.

Lastly, a fourth exemplary cost function is provided in Equations (9) below. The cost function (9) involves both 3^{rd} and 4^{th} Gen data pre-decomposition and weight with a predetermined information matrix such as Fisher information matrix.

$$ψ ( c ) = {\sum }_{{jnn}^{′}} {I}_{{nn}^{′}} ( j ) ( {\sum }_{i} {a}_{ji} {c}_{n} ( i ) - {l}_{n}^{( M )} ( j ) ) ( {\sum }_{{i}^{′}} {a}_{ji} {c}_{{n}^{′}} ( {i}^{′} ) - {l}_{{n}^{′}}^{( M )} ( j ) ) + {\sum }_{jn} \frac{1}{{σ}_{jn}^{2}} {( {\sum }_{i} {A}_{ji} {c}_{n} ( i ) - {L}_{nM}^{( M )} ( j ) )}^{2} + wV ( c ) \qquad ( 9 )$$

Fisher information matrix is defined by the following terms:

$${I}_{{nm}^{′}} ( j ) = {\sum }_{E} \frac{1 \partial {N}_{j} ( E ) \partial {N}_{j} ( E )}{{N}_{j} ( E ) \partial {l}_{n} ( j ) \partial {l}_{{n}^{′}} ( j )}$$

Nⱼ(E) is a photon count in an energy bin E for a ray path j.
where c is a basis image vector, σⱼₙ² is variance of Lₙ^{(M)}(j), aⱼᵢ is a system matrix for a 4^{th}-Gen scanner for acquiring the sparse views of the spectral data, Aⱼᵢ is a system matrix for a 3^{rd}-Gen scanner for acquiring the full views of the intensity data, c^{′}ₙ(i^{′}) is material basis images, V(c) is a regularization term. lₙ^{(M)}(j) is material length for basis n along a ray j from the sparse views of the spectral data after decomposition (measured 4^{th} Gen data after decomposition) while Lₙ^{(M)}(j) is a re-projected material length for the measured 3^{rd} Gen data after decomposition. In one embodiment, a weight w for the regularization term is set to zero (w=0) for turning off the regularization term as used in one embodiment of the current invention.

$${L}_{n}^{( M )} ( j ) is decided by$$

$${\sum }_{n = 1}^{N} {L}_{n}^{( M )} ( j ) {\overline{μ}}_{nM} - {g}_{M} ( j ) - {g}_{M}^{( BH )} ( L ) = 0$$

$${\sum }_{n = 1}^{N} [ {L}_{n}^{( M )} ( j ) - {\sum }_{i} {A}_{ji} {c}_{n} ( i ) ] {μ}_{n} ( E ) = 0$$

Wherein g_{M}(j) is the full views of the intensity data (measured 3rd Gen data), g_{M}^{(BH)}(L) is a beam-hardening correction term with L being a material length vector of the basis material, μ̅ₙ is a basis function, usually a linear attenuation coefficient of material n at energy E.

As described above with respect to the step S300, a process of reconstructing an improved image forms a predetermined cost function based upon the 3^{rd} and 4^{th} Gen data according to the current invention. Although a penalty or regularization term is optionally included, it can be weighted as shown in the above embodiments. In addition, a process according to the current invention is not limited to the above illustrated cost functions, and another cost function is optionally utilized in ultimately reconstructing an improved image based upon sparse views of the spectral data and full views of the intensity data according to one process of the current invention. The ratio between a number of the sparse views in the spectral data and a number of the full views is the intensity data is also not limited to a particular value and has an appropriate range based upon multiple factors such as a clinical application and so on.

Furthermore, the predetermined cost function is minimized to find the spectral images as will be described with respect to steps S400 and S500. In the step S400, a predetermined cost function based upon the 3^{rd} and 4^{th} Gen data is minimized with a predetermined iterative procedure in one process of improving an image according to the current application. In general, the basis image cₙ(i) is updated during the iterative procedure as seen in Equation (10) below:

$${c}_{n} ( i ) = {c}_{n}^{( 0 )} ( i ) - \frac{\frac{\partial ψ ( {c}^{( 0 )} )}{\partial {C}_{n} ( i )}}{{\sum }_{{i}^{′} {n}^{′}} \frac{{\partial }^{2} ψ ( {c}^{( 0 )} )}{\partial {C}_{n} ( i ) \partial {C}_{{n}^{′}} ( {i}^{′} )}} \qquad ( 10 )$$

Equation (10) is an exemplary algorithm that has been derived according to the cost function as provided in Equation (1) and is not limited to this particular equation. In fact, there are other equations corresponding to the above exemplary cost functions as illustrated by Equations (3), (6) and (9).

The above described iterative procedure is repeated in the steps S400 and S500 in order to find the spectral images including a positivity constraint in one process of improving an image utilizing the 3^{rd} and 4^{th} Gen data according to the current invention. The iteration is repeated if a predetermined condition has not yet been met as it is determined in the step S500. On the other hand, the iteration is terminated when the predetermined condition is reached as it is determined in the step S500. The predetermined condition for termination is not limited to a particular condition and includes a number of conditions such as a predetermined maximal number of iterations or a difference in a certain value between the current instance and the previous instance. The predetermined terminating condition also optionally depends upon other conditions such as a clinical application.

As described above, the 3^{rd} Gen data and 4^{th} Gen data are obtained in the same gantry. In general, the images reconstructed from the 3^{rd} Gen data have no spectral information. On the other hand, although the images reconstructed from the 4^{th} Gen data have spectral information, the reconstructed image may contain aliasing artifacts due to the sparse views if the 4^{th} Gen data is processed by itself. According to the current invention, a process combines the two datasets of the 3^{rd} Gen data and 4^{th} Gen data and processes the combined data with an iterative procedure. In the above described 3^{rd}+4^{th} Gen spectral CT, the projection data from the sparse photon counting detectors provides spectral information while the projection data from the energy integration detectors provides spectral integrated information. The combined data has improved images with spectral information, and the improved images are substantially free from aliasing artifacts even though the spectral data is gathered from sparse detectors.

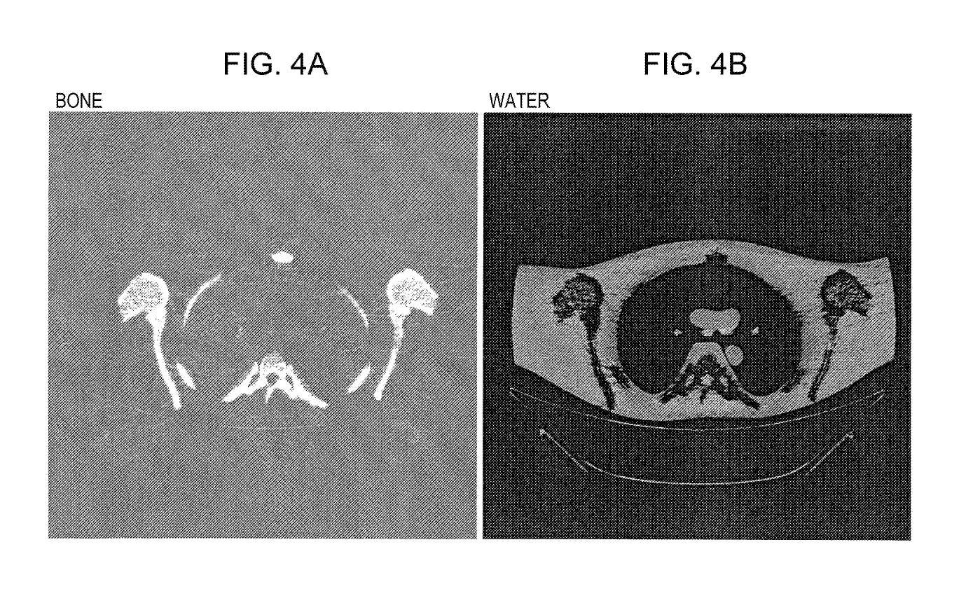

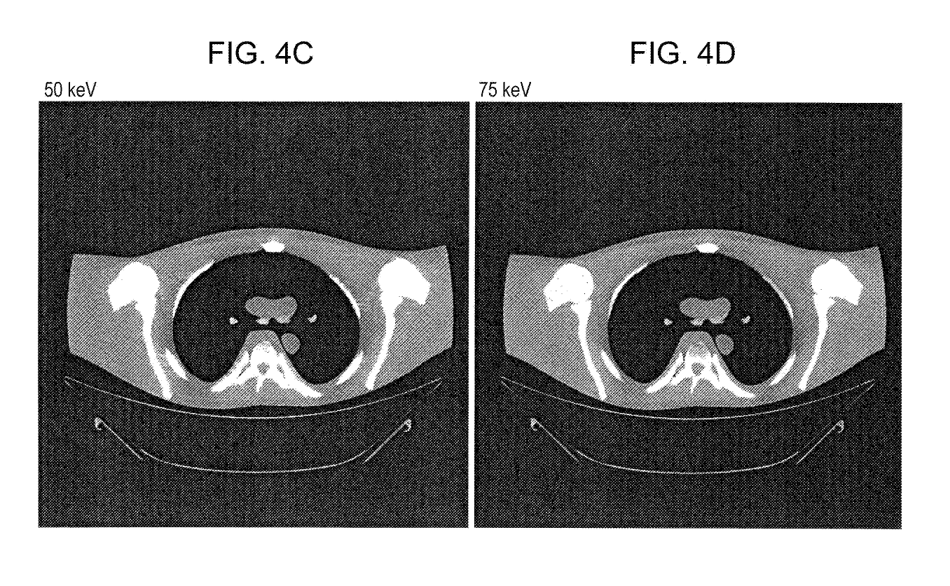

Now referring to FIGS. 4A through 4D, images illustrate some result of the above described process utilizing the combined data of sparse spectral data and full integration data of a predetermined torso phantom according to the current invention. The full integration data is based upon 140 kVP and 1200 views of the circular scans. The sparse spectral data is abased upon 100 kVP and 75 views. FIG. 4A is a basis image for bone while FIG. 4B is a basis image for water. FIG. 4C is a monochromatic image at 50 keV while FIG. 4D is a monochromatic image at 75 keV.

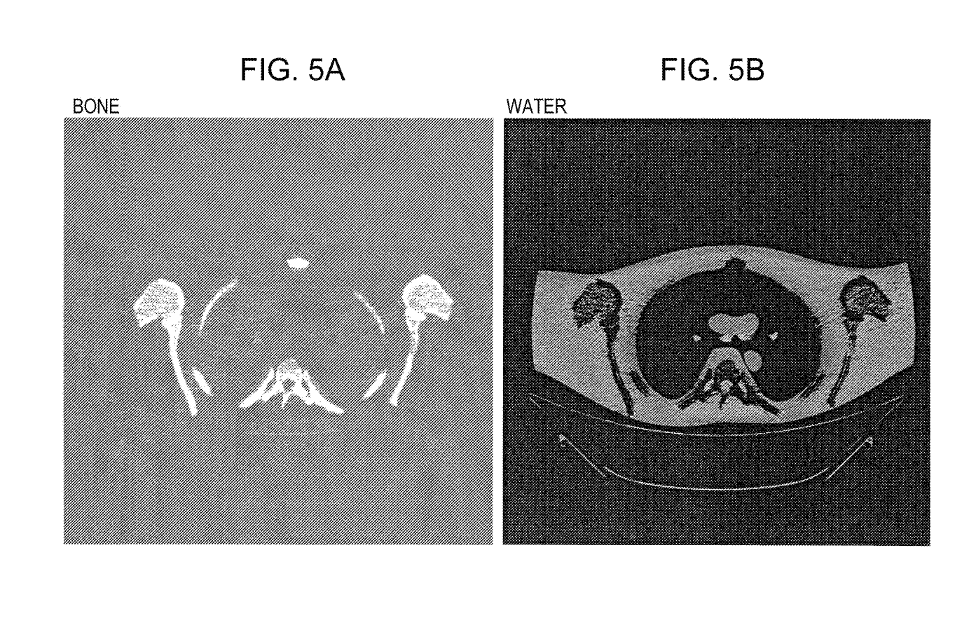

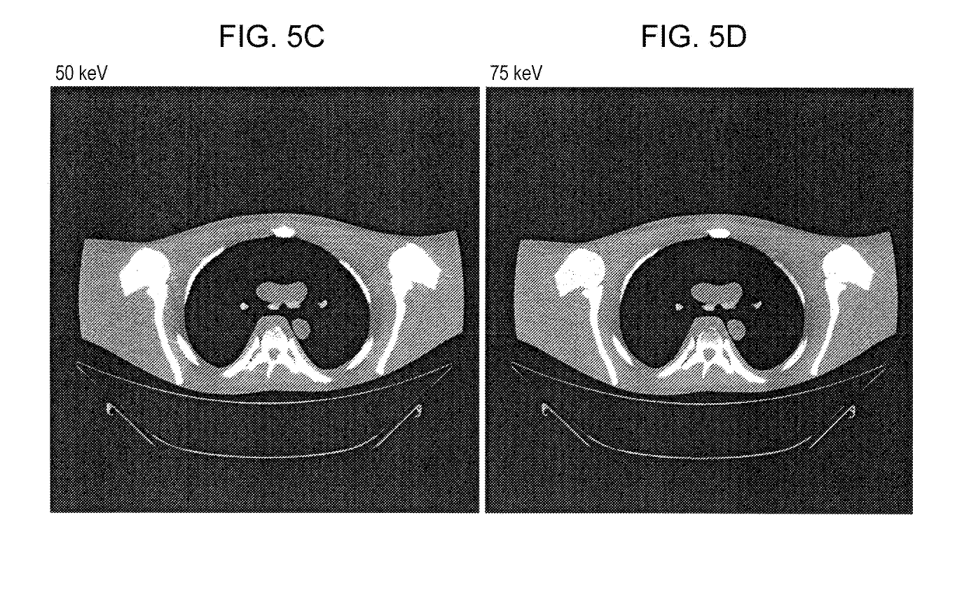

Now referring to FIGS. 5A through 5D, images illustrate some result of the above described process utilizing the combined data of sparse spectral data and full integration data of a predetermined torso phantom according to the current invention. The full integration data is based upon 140 kVP and 1200 views of the circular scans. The sparse spectral data is abased upon 100 kVP and 150 views. FIG. 5A is a basis image for bone while FIG. 5B is a basis image for water. FIG. 5C is a monochromatic image at 50 keV while FIG. 5D is a monochromatic image at 75 keV.

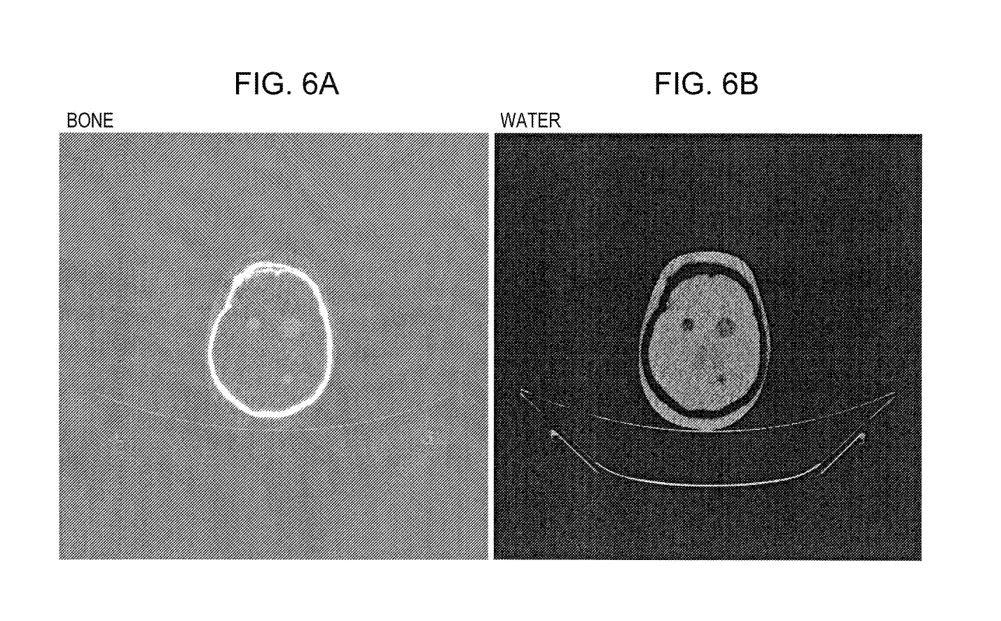

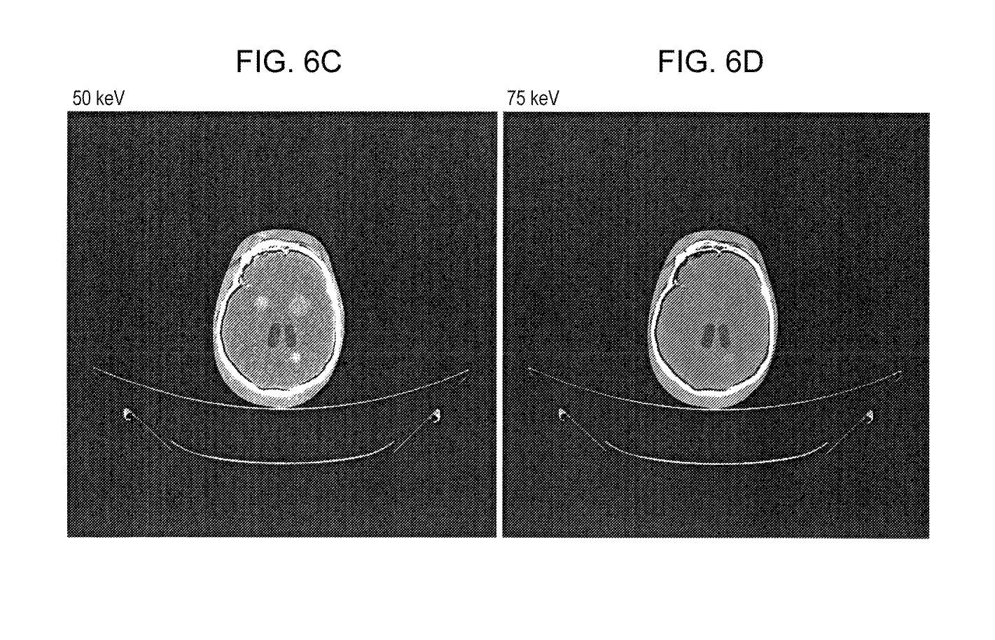

Now referring to FIGS. 6A through 6D, images illustrate some result of the above described process utilizing the combined data of sparse spectral data and full integration data of a predetermined head phantom according to the current invention. The full integration data is based upon 140 kVP and 1200 views of the circular scans. The sparse spectral data is abased upon 100 kVP and 75 views. FIG. 6A is a basis image for bone while FIG. 6B is a basis image for water. FIG. 6C is a monochromatic image at 50 keV while FIG. 6D is a monochromatic image at 75 keV.

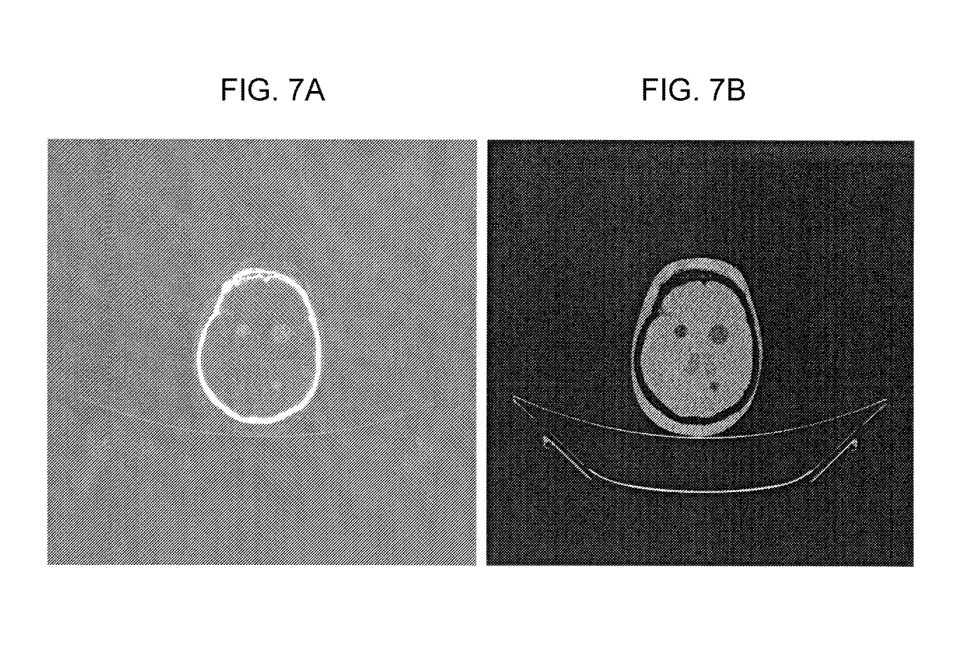

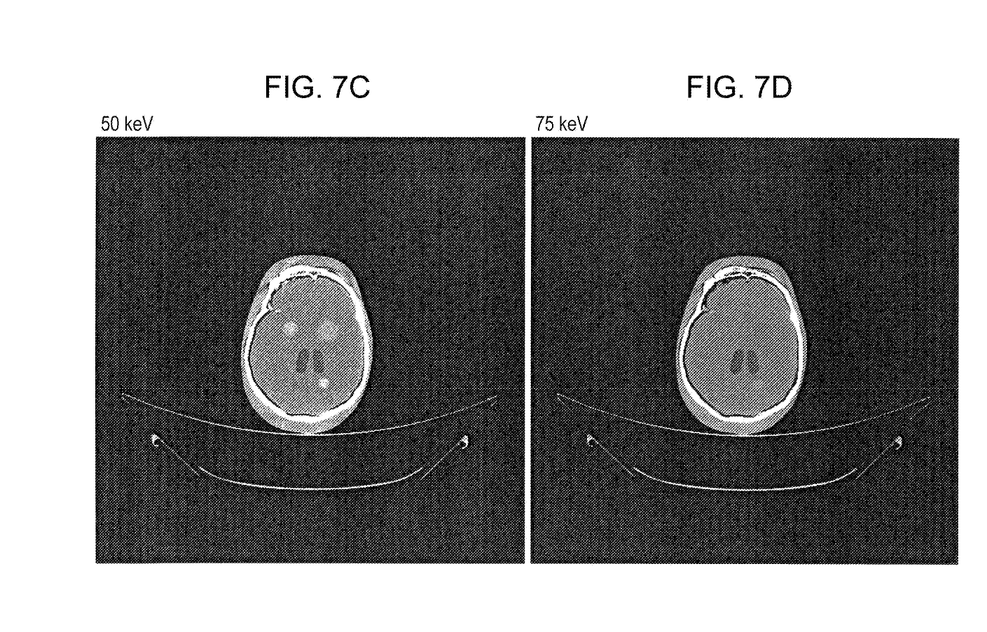

Now referring to FIGS. 7A through 7D, images illustrate some result of the above described process utilizing the combined data of sparse spectral data and full integration data of a predetermined head phantom according to the current invention. The full integration data is based upon 140 kVP and 1200 views of the circular scans. The sparse spectral data is abased upon 100 kVP and 150 views. FIG. 7A is a basis image for bone while FIG. 7B is a basis image for water. FIG. 7C is a monochromatic image at 50 keV while FIG. 7D is a monochromatic image at 75 keV.

What is claimed is:

1. A method of reconstructing an image, comprising:
acquiring full views of intensity data of an object;
acquiring sparse views of spectral data of the object; and
reconstructing an image of the object based upon the full views of the intensity data and the sparse views of the spectral data.

2. The method of reconstructing an image according to claim 1 wherein the image is reconstructed based upon a predetermined iterative reconstruction algorithm.

3. The method of reconstructing an image according to claim 1 wherein said reconstructing step further comprises:
forming a predetermined cost function based upon the full views of the intensity data and the sparse views of the spectral data; and
minimizing the predetermined cost function.

4. The method of reconstructing an image according to claim 1 wherein the predetermined cost function includes:
$$\begin{matrix}ψ ( c ) = {\sum }_{jn} \frac{1}{{σ}_{jn}^{2}} {( {l}_{n} ( j ) - {l}_{n}^{( M )} ( j ) )}^{2} + {\sum }_{j} \frac{1}{{σ}_{j}^{2}} {( {\sum }_{n = 1}^{N} {L}_{n} ( j ) {\overline{μ}}_{nM} - {g}_{M} ( j ) - {g}_{M}^{( BH )} ( L ) )}^{2} + wV ( c ) \\ {l}_{n} ( j ) = {\sum }_{i} {a}_{ji} {c}_{n} ( i ) \\ {L}_{n} ( j ) = {\sum }_{i} {A}_{ji} {c}_{n} ( i )\end{matrix}$$
wherein c is a basis image vector, σⱼₙ² is variance of lₙ^{(M)}(j) while σⱼ² is variance of g_{M}(j), μ̅_{nM} is an average linear attenuation coefficient over a spectrum of the intensity data for a basis n, aⱼᵢ is a system matrix for a scanner for acquiring the sparse views of the spectral data, Aⱼᵢ is a system matrix for a scanner for acquiring the full views of the intensity data, cₙ(i) is material basis images, V(c) is a regularization term, g_{M}(j) is the full views of the intensity data, g_{M}^{(BH)}(L) is a beam-hardening correction term with L being a material length vector of a basis material, lₙ^{(M)}(j) is a material length for a basis n along a ray j from the sparse views of the spectral data after data decomposition, lₙ(j) is a re-projected material length for the spectral data while Lₙ(j) is a re-projected material length for the intensity data.

5. The method of reconstructing an image according to claim 4 wherein the cost function is optionally minimized with an iterative algorithm using one of polar coordinates, a system matrix, normalization, initialization, update, positivity constraint and penalty.

6. The method of reconstructing an image according to claim 1 wherein the spectral data includes information across a full range of energy levels.

7. The method of reconstructing an image according to claim 1 wherein the spectral data includes information on dual energy levels.

8. The method of reconstructing an image according to claim 1 wherein the sparse views include approximately 75 views.

9. The method of reconstructing an image according to claim 1 wherein the full views include approximately 1200 views.

10. The method of reconstructing an image according to claim 1 wherein the spectral data includes photo counting information for a predetermined number of energy bins.

11. The method of reconstructing an image according to claim 1 wherein the spectral data and the intensity data are respectively acquired with a source radiation at a predetermined different energy level.

12. The method of reconstructing an image according to claim 1 wherein the full views of the intensity data and the sparse views of the spectral data of the object are simultaneously acquired.

13. The method of reconstructing an image according to claim 1 wherein the full views of the intensity data and the sparse views of the spectral data of the object are sequentially acquired.

14. A method of reconstructing an image, comprising:
acquiring full views of intensity data of an object;
acquiring sparse views of spectral data of the object; and
reconstructing an image of the object based upon the full views of the intensity data and the sparse views of the spectral data using an iterative reconstruction algorithm to minimize a predetermined cost function that includes
$$\begin{matrix}ψ ( c ) = {\sum }_{jn} \frac{1}{{σ}_{jn}^{2}} {( {l}_{n} ( j ) - {l}_{n}^{( M )} ( j ) )}^{2} + {\sum }_{j} \frac{1}{{σ}_{j}^{2}} {( {\sum }_{n = 1}^{N} {L}_{n} ( j ) {\overline{μ}}_{nM} - {g}_{M} ( j ) - {g}_{M}^{( BH )} ( L ) )}^{2} + wV ( c ) \\ {l}_{n} ( j ) = {\sum }_{i} {a}_{ji} {c}_{n} ( i ) \\ {L}_{n} ( j ) = {\sum }_{i} {A}_{ji} {c}_{n} ( i )\end{matrix}$$
wherein c is a basis image vector, σⱼₙ² is variance of lₙ^{(M)}(j) while σⱼ² is variance of g_{M}(j), μ̅_{nM} is an average linear attenuation coefficient over a spectrum of the intensity data for a basis n, aⱼᵢ is a system matrix for a scanner for acquiring the sparse views of the spectral data, Aⱼᵢ is a system matrix for a scanner for acquiring the full views of the intensity data, cₙ(i) is material basis images, V(c) is a regularization term, g_{M}(j) is the full views of the intensity data, g_{M}^{(BH)}(L) is a beam-hardening correction term with L being a material length vector of a basis material, lₙ^{(M)}(j) is a material length for a basis n along a ray j from the sparse views of the spectral data after data decomposition, lₙ(j) is a re-projected material length for the spectral data while Lₙ(j) is a re-projected material length for the intensity data.

15. A system for reconstructing an image, comprising:
at least one intensity data acquiring device for acquiring full views of intensity data of an object;
at least one spectral data acquiring device for acquiring sparse views of spectral data of the object; and
a reconstruction device ultimately connected to said intensity data acquiring device and said spectral data acquiring device for reconstructing an image of the object based upon the full views of the intensity data and the sparse views of the spectral data.

16. The system for reconstructing an image according to claim 15 wherein said reconstruction device reconstructs the image based upon a predetermined iterative reconstruction algorithm.

17. The system for reconstructing an image according to claim 15 wherein said reconstruction device forms a predetermined cost function based upon the full views of the intensity data and the sparse views of the spectral data, said reconstruction device minimizing the predetermined cost function.

18. The system for reconstructing an image according to claim 15 wherein the predetermined cost function includes:
$$\begin{matrix}ψ ( c ) = {\sum }_{jn} \frac{1}{{σ}_{jn}^{2}} {( {l}_{n} ( j ) - {l}_{n}^{( M )} ( j ) )}^{2} + {\sum }_{j} \frac{1}{{σ}_{j}^{2}} {( {\sum }_{n = 1}^{N} {L}_{n} ( j ) {\overline{μ}}_{nM} - {g}_{M} ( j ) - {g}_{M}^{( BH )} ( L ) )}^{2} + wV ( c ) \\ {l}_{n} ( j ) = {\sum }_{i} {a}_{ji} {c}_{n} ( i ) \\ {L}_{n} ( j ) = {\sum }_{i} {A}_{ji} {c}_{n} ( i )\end{matrix}$$
wherein c is a basis image vector, σⱼₙ² is variance of lₙ^{(M)}(j) while σⱼ² is variance of g_{M}(j), μ̅_{nM} is an average linear attenuation coefficient over a spectrum of the intensity data for a basis n, aⱼᵢ is a system matrix for a scanner for acquiring the sparse views of the spectral data, Aⱼᵢ is a system matrix for a scanner for acquiring the full views of the intensity data, cₙ(i) is material basis images, V(c) is a regularization term, g_{M}(j) is the full views of the intensity data, g_{M}^{(BH)}(L) is a beam-hardening correction term with L being a material length vector of a basis material, lₙ^{(M)}(j) is a material length for a basis n along a ray j from the sparse views of the spectral data after data decomposition, lₙ(j) is a re-projected material length for the spectral data while Lₙ(j) is a re-projected material length for the intensity data.

19. The system for reconstructing an image according to claim 18 wherein the cost function is optionally minimized with an iterative algorithm using one of polar coordinates, a system matrix, normalization, initialization, update, positivity constraint and penalty.

20. The system for reconstructing an image according to claim 15 wherein the spectral data includes information across a full range of energy levels.

21. The system for reconstructing an image according to claim 15 wherein the spectral data includes information on dual energy levels.

22. The system for reconstructing an image according to claim 15 wherein the sparse views include approximately 75 views.

23. The system for reconstructing an image according to claim 15 wherein the full views include approximately 1200 views.

24. The system for reconstructing an image according to claim 15 wherein the spectral data includes photo counting information for a predetermined number of energy bins.

25. The system for reconstructing an image according to claim 15 wherein the spectral data and the intensity data are respectively acquired with a source radiation at a predetermined different energy level.

26. The system for reconstructing an image according to claim 15 wherein said intensity data acquiring device acquires the full views of the intensity data while said spectral data acquiring device simultaneously acquires the sparse views of the spectral data of the object.

27. The system for reconstructing an image according to claim 15 wherein said intensity data acquiring device acquires the full views of the intensity data, said spectral data acquiring device independently acquires the sparse views of the spectral data of the object.

28. A system for reconstructing an image, comprising:
an intensity data acquiring device for acquiring full views of intensity data of an object;
a spectral data acquiring device for acquiring sparse views of spectral data of the object; and
a reconstruction device ultimately connected to said intensity data acquiring device and said spectral data acquiring device for reconstructing an image of the object based upon the full views of the intensity data and the sparse views of the spectral data using an iterative reconstruction algorithm to minimize a predetermined cost function that includes
$$\begin{matrix}ψ ( c ) = {\sum }_{jn} \frac{1}{{σ}_{jn}^{2}} {( {l}_{n} ( j ) - {l}_{n}^{( M )} ( j ) )}^{2} + {\sum }_{j} \frac{1}{{σ}_{j}^{2}} {( {\sum }_{n = 1}^{N} {L}_{n} ( j ) {\overline{μ}}_{nM} - {g}_{M} ( j ) - {g}_{M}^{( BH )} ( L ) )}^{2} + wV ( c ) \\ {l}_{n} ( j ) = {\sum }_{i} {a}_{ji} {c}_{n} ( i ) \\ {L}_{n} ( j ) = {\sum }_{i} {A}_{ji} {c}_{n} ( i )\end{matrix}$$
wherein c is a basis image vector, σⱼₙ² is variance of lₙ^{(M)}(j) while σⱼ² is variance of g_{M}(j), μ̅_{nM} is an average linear attenuation coefficient over a spectrum of the intensity data for a basis n, aⱼᵢ is a system matrix for a scanner for acquiring the sparse views of the spectral data, Aⱼᵢ is a system matrix for a scanner for acquiring the full views of the intensity data, cₙ(i) is material basis images, V(c) is a regularization term, g_{M}(j) is the full views of the intensity data, g_{M}^{(BH)}(L) is a beam-hardening correction term with L being a material length vector of a basis material, lₙ^{(M)}(j) is a material length for a basis n along a ray j from the sparse views of the spectral data after data decomposition, lₙ(j) is a re-projected material length for the spectral data while Lₙ(j) is a re-projected material length for the intensity data.
